(19) United States
(12) Patent Application Publication
(10) Pub. No.: US 2015/0359471 A1
(43) Pub. Date: Dec. 17, 2015

(54) COMPOSITE MEDICAL EXAMINATION DEVICE

(71) Applicant: Bryant Sperry, Murray, UT (US)

(72) Inventor: Bryant Sperry, Murray, UT (US)

(21) Appl. No.: 14/738,571

(22) Filed: Jun. 12, 2015

Related U.S. Application Data
(60) Provisional application No. 62/011,361, filed on Jun. 12, 2014.

(51) Int. Cl. A61B 5/1455; A61B 7/04; A61B 5/024; A61B 5/00

(52) U.S. Cl. CPC A61B 5/14552; A61B 5/742; A61B 7/04; A61B 5/02427

(57) ABSTRACT

A medical examination device can include a stethoscope having a chest piece coupled to at least one ear piece via a yoke. The chest piece can have an acoustic portion configured to interface with a subject's body to provide sounds produced in the body to the ear pieces. The medical examination device can also include a pulse oximeter associated with an oximeter portion of the chest piece. The pulse oximeter can have a light emitter and a photodetector to measure oxygen saturation of the subject's blood. In addition, the medical examination device can include a display for displaying information of the stethoscope and/or the pulse oximeter.

### RELATED APPLICATION

This application claims priority to U.S. Provisional Application No. 62/011,361, filed Jun. 12, 2014, which is incorporated herein by reference.

### BACKGROUND

A conventional pulse oximeter, which is pervasive in the healthcare industry, measures blood oxygen levels using a fingertip clip that transmits and receives IR light through a finger. Such contact with patents' fingertips can be problematic, however, because disease is commonly transmitted via contact with fingers, and is therefore a very common source of infection in hospital and patient care facilities. The mortality rate of hospital-acquired infections (HAI) is nearing 7% according to the CDC. HAI presents not only health concerns, but financial concerns, as well. For example, the average cost of treatment, per patient, for HAI is about $10,000.

In addition, many hospital patients have poor vascularity in their extremities, which can significantly hinder obtaining a blood oxygen measurement from the fingertips. Moreover, in many cases, access to fingertips is obstructed by IV tubes, which can make obtaining a blood oxygen measurement difficult for the patient and caregiver alike.

### SUMMARY

Thus, there is a need for a medical examination device capable of obtaining blood oxygen measurements conveniently from a location other than fingers or fingertips. Accordingly, a medical examination device and associated systems are provided. Such a device can comprise a stethoscope having a chest piece coupled to at least one ear piece via a yoke. The chest piece can have an acoustic portion configured to interface with a subject's body to provide sounds produced in the body to the ear pieces. The medical examination device can also comprise a pulse oximeter associated with an oximeter portion of the chest piece. The pulse oximeter can have a light emitter and a photodetector to measure oxygen saturation of the subject's blood. In addition, the medical examination device can include a display for displaying information of the stethoscope and/or the pulse oximeter.

Furthermore, a medical examination system in accordance with the principles herein can comprise a medical examination device including a stethoscope having a chest piece coupled to at least one ear piece via a yoke. The chest piece can have an acoustic portion configured to interface with a body of a subject to provide sounds produced in the body to the ear pieces. The medical examination device can have a pulse oximeter associated with an oximeter portion of the chest piece. The pulse oximeter can have a light emitter and a photodetector to measure oxygen saturation of the subject's blood. The medical examination device can further include a display for displaying information of the stethoscope and/or the pulse oximeter. Additionally, the medical examination device can include a battery to provide power for the medical examination device. The system can also comprise a storage device having a chamber to receive the medical examination device, and a battery charger to charge the battery of the medical examination device when disposed in the chamber.

In addition, another medical examination device in accordance with the principles herein can comprise an ear thermometer having a probe portion configured to be disposed in an ear of a subject, and an infrared detector associated with the probe portion to receive thermal radiation from the ear to facilitate determination of a temperature of the subject. The medical examination device can also comprise a pulse oximeter associated with the probe portion. The pulse oximeter can have a light emitter and a photodetector to measure oxygen saturation of the subject's blood. In addition, the medical examination device can comprise a display for displaying information of the ear thermometer and/or the pulse oximeter.

### BRIEF DESCRIPTION OF THE DRAWINGS

FIG. 1 illustrates a composite stethoscope medical examination device, in accordance with an example of the present disclosure.

FIG. 2 illustrates a composite stethoscope medical examination device, in accordance with another example of the present disclosure.

FIG. 3A illustrates a pulse oximeter portion in accordance with one example of the present disclosure.

FIG. 3B illustrates a flexible member through which multiple wires can pass in accordance with examples of the present disclosure.

FIG. 4A illustrates a storage device for a medical examination device, showing a closed configuration in accordance with an example of the present disclosure.

FIG. 4B illustrates the storage device of FIG. 4A showing an open configuration.

FIG. 5 illustrates a medical examination device including an ear thermometer, in accordance with yet another example of the present disclosure.

FIG. 6 illustrates a mounting structure to secure a medical examination device on a mobile caddy, in accordance with an example of the present disclosure.

FIG. 7 illustrates a medical examination device, in accordance with still another example of the present disclosure.

FIG. 8 is a schematic of a medical examination device having a compliant diaphragm and an attachment loop in accordance with still another example of the present disclosure.

These figures are provided merely for convenience in describing specific embodiments of the invention. Alteration in dimension, materials, and the like, including substitution, elimination, or addition of components can also be made consistent with the following description and associated claims. Reference will now be made to the exemplary embodiments illustrated, and specific language will be used herein to describe the same. It will nevertheless be understood that no limitation of the scope of the invention is thereby intended.

### DETAILED DESCRIPTION

Reference will now be made to certain examples, and specific language will be used herein to describe the same. Examples discussed herein set forth a medical examination device and associated systems that can obtain blood oxygen measurements conveniently from a location other than fingers or fingertips.

With the general embodiments set forth above, it is noted that when describing a medical examination device, or related systems and/or methods, each of these descriptions are considered applicable to the other, whether or not they are explicitly discussed in the context of that embodiment. For example, in discussing the medical examination device per se, system and/or method embodiments are also included in such discussions, and vice versa.

It is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a light emitter” includes one or more of such light emitters and reference to “the photodetector” includes one or more of such photodetectors.

In describing and claiming the present invention, the following terminology will be used in accordance with the definitions set forth below.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Any steps recited in any method or process claims may be executed in any order and are not limited to the order presented in the claims unless otherwise stated. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

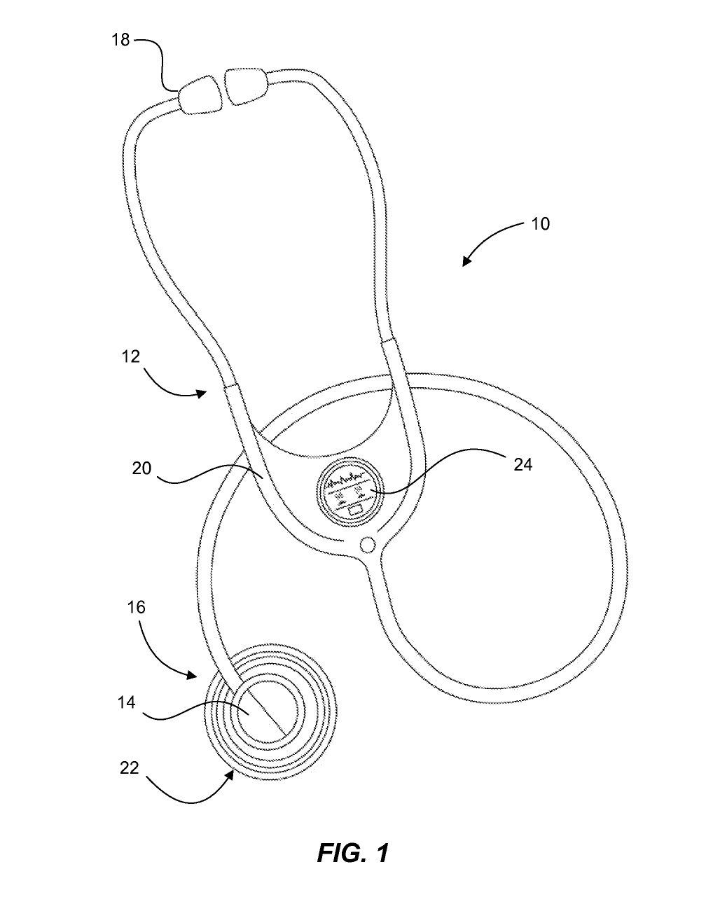

Illustrated in FIG. 1 is a composite medical examination device 10 including a stethoscope portion 12 and a pulse oximeter portion 14. In accordance with one example of the present disclosure, the medical examination device can comprise a stethoscope, which can be any suitable type of stethoscope, such as, but not limited to, a traditional acoustic stethoscope, an electronic stethoscope, or a Doppler stethoscope. The general construction of the stethoscope can be typical of traditional stethoscopes, in that the stethoscope can have a chest piece 16 coupled to a pair of ear pieces 18 via a yoke 20. However, a single ear piece can also be used. Optionally, the ear pieces can be disposable and replaceable. The chest piece 16 can have an acoustic portion 22 configured to interface with a subject's body to provide sounds produced in the body which can be conveyed to the ear piece(s). The acoustic portion can include a diaphragm and/or a microphone or other acoustically responsive sensor. Thus, in one aspect, the stethoscope can electronically provide sounds produced in the body to the ear pieces, such as by wired and/or wireless transmission.

The medical examination device 10 can also include a pulse oximeter portion 14 integrated with the stethoscope 12. For example, the pulse oximeter portion 14 can be associated with the chest piece 16 of the stethoscope 12. The pulse oximeter can measure the amount of saturated hemoglobin in tissue capillaries by transmitting a beam of light through the tissue to a receiver. Because the amount of saturated hemoglobin alters the wavelengths of the transmitted light, analysis of the received light can be translated into a percentage of oxygen saturation (SO₂) of the blood. Translation of such data to calculate oxygen saturation can be performed using known correlations. Non-limiting examples of suitable pulse oximetry correlations include wavelet transform analysis, Fourier transforms, and the like. In one example, the wavelet transform of a signal x(t) is defined as:

$$T ( a , b ) = \frac{1}{\sqrt{a}} {\int }_{- ∞}^{+ ∞} x ( t ) {ψ}^{*} ( \frac{t - b}{a} ) d t$$

where t is time, a is scale parameter where a>0, b is position, and ψ* is the complex conjugate of the wavelet. Regardless of the specific data analysis, additional methods can include, but are not limited to, spO2 to fraction of inspired oxygen (fiO2) ration, dual wavelength signals, The pulse oximeter can include typical features of a pulse oximeter. For example, the pulse oximeter can have one or more light emitters, such as an IR and/or near IR LED, and one or more photodetectors to measure oxygen saturation of the subject's blood. In one aspect, one or more photodetectors can function independently to provide improved accuracy and fault tolerances.

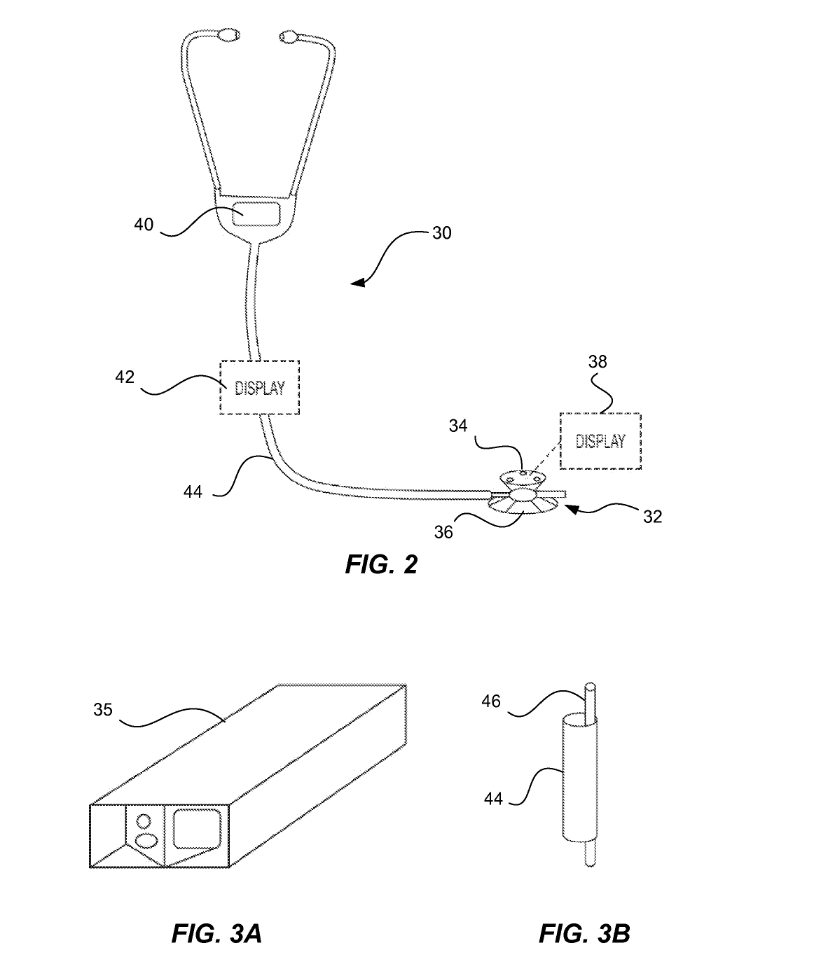

In one aspect, the light emitter and the photodetector can be proximate one another and can be included in an oximeter portion 14 of the chest piece 16. For example, the oximeter portion 14 can be on a side of the chest piece 16 opposite the acoustic portion 22 and the light emitter and the photodetector can be proximate to one another, as shown in FIG. 1 and FIG. 2. In this configuration, the pulse oximeter can operate by reflectance of the transmitted light from within the tissue to the photodetector, which can provide for universal application of the pulse oximeter, such as the chest, forehead, and feet. This is in contrast to a typical pulse oximeter that is in contact with a subject's finger and operates by transmitting light from a light source on one side of the finger and through the finger where the light is received by a photodetector on an opposite side of the finger. Thus, the pulse oximeter can be integrated with the chest piece and therefore used in a manner typical of the chest piece, which is to be placed in contact with a subject's chest, back, stomach, etc.

In addition, the medical examination device can include a display 24 for displaying information of the stethoscope 12 and/or the pulse oximeter 14. In one aspect, the display 24 can be associated with the yoke 20. One or more batteries can be included to provide power for the stethoscope, pulse oximeter, the display, and/or any electronic circuitry, processor, transmitter, receiver, etc. incorporated into the medical examination device. Regardless, the display can be in electrical communication with the stethoscope and/or pulse oximeter. In one alternative, the pulse oximeter can include a processor which translates light frequency data into pulse oximetry values. In yet another alternative, the display can include a secondary processor which receives signals from the stethoscope and/or pulse oximeter and provides a user configurable output to the display. For example, the display can be configured to toggle between heart rate, acoustic profiles (e.g. sound wave graph), oximetry data, and the like, or can be configured to allow adjustment of brightness, data collection and/or refresh rates, etc.

In one aspect, the medical examination device can further include a timer and an alert feature to facilitate determining a heart rate of the subject by a user. For example, the timer can be configured to provide an alert at 15 seconds and at 30 seconds to facilitate determining a heart rate by a user of the device. The alert feature can be configured to provide any suitable type of alert, such as an audible alert, a visual alert, and/or a haptic alert. Thus, in one aspect, the alert feature can cause a vibration at 15 seconds and at 30 seconds to enable the user to determine a heart rate without looking at a clock. In another aspect, the alert feature can cause an audible voice to alert the user at the beginning of a timing period, at 15 seconds, and again at 30 seconds.

In one aspect, the medical examination device can be configured to determine a heart rate of the subject by electronic means, such as by processing sound data obtained from the heart or with one or more electrodes for electrocardiography. The medical examination device can also include any suitable button, switch, touch screen, etc. to facilitate operation and control of the device by a user, such as powering the device on/off, selecting an operation mode, and selecting a display mode.

FIG. 2 illustrates a medical examination device 30 in accordance with another example of the present disclosure. The medical examination device 30 of FIG. 2 is similar in many respects to the medical examination device 10 of FIG. 1. In this case, however, the chest piece 32 can include a pulse oximeter portion 34 having a pulse oximeter 35 (detailed in FIG. 3A) that extends outward and away from the acoustic portion 36 to enable the chest piece 32 to have additional uses. For example, the chest piece can include an acoustic portion that has both a diaphragm portion and a bell portion typical of traditional acoustic stethoscopes. Thus, in one aspect, the pulse oximeter portion can be oriented opposite the acoustic portion. In an alternative aspect, the pulse oximeter portion can be oriented on a common side adjacent to or within the acoustic portion.

In one aspect, the chest piece can further include one or more electrocardiogram leads to provide heart rate and/or rhythm information. As generally illustrated in FIG. 2, a display 38 and 40 can be associated with one or both the chest piece and at the yoke. Alternately, the display 42 can be associated with a flexible member 44 extending between the chest piece 32 and the yoke 40. Wires 46 connecting the pulse oximeter, electrocardiogram leads, etc. to the display and/or other electronic circuitry can run through the flexible member 44, as shown in FIG. 3B.

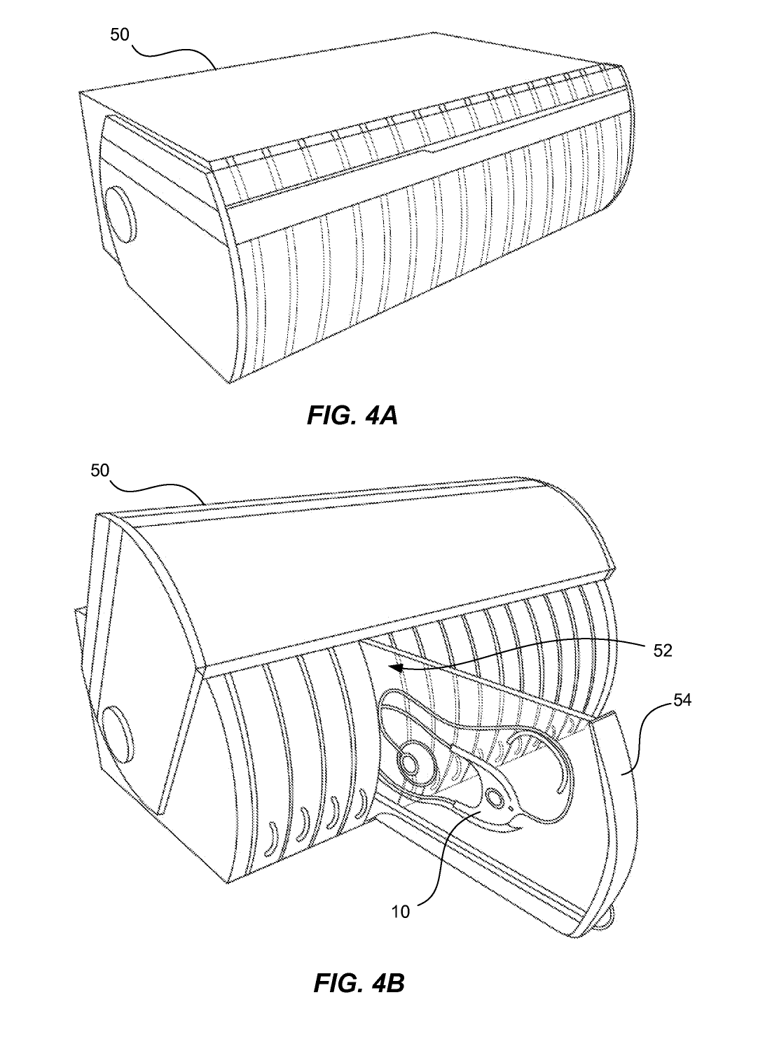

FIGS. 4A and 4B illustrate a storage device 50 for the medical examination device 10 in accordance with an example of the present disclosure. Although the medical examination device shown in the figure is similar to the medical examination device of FIG. 1, the storage device can be configured to accommodate any suitable medical examination device, in any combination. For example, the storage device can have a chamber 52 to receive the medical examination device 10. In one aspect, the storage device 50 can include a support structure 54 for the medical examination device configured to slide into and out of the chamber 52. In another aspect, the storage device can have a plurality of chambers to accommodate a plurality of medical devices, which may be of any suitable type or configuration. In addition, the storage device can include a battery charger to charge a battery of the medical examination device when disposed in the chamber. The battery charger can be configured to couple with the medical examination device via a wire or cord, or via a wireless coupling, such as utilizing inductive charging to charge the battery of the medical examination device. In one aspect, the storage device can include a sterilizing unit which is operable to sanitize the medical examination device when disposed in the chamber. In one example, the sterilizing unit can be an ultraviolet light source oriented to expose the medical examination device within the chamber to ultraviolet light. Alternative sterilizing units can include lasers, acoustic (ultrasonic) vibration, disinfectant spray, autoclave, dry heat, and the like.

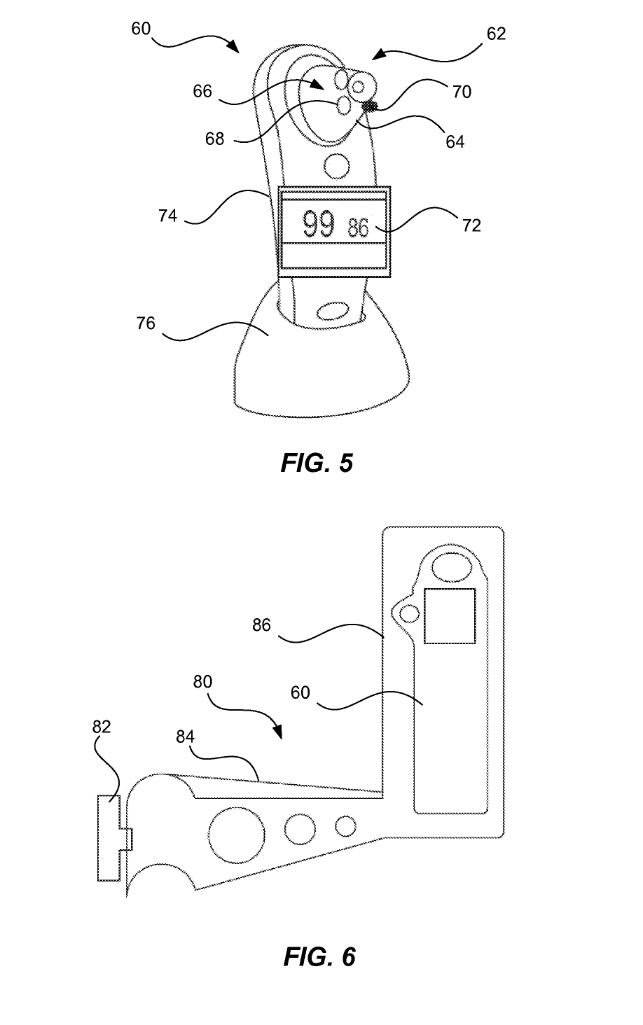

FIG. 5 illustrates a medical examination device in accordance with another example of the present disclosure. The medical examination device 60 can be a composite ear thermometer and pulse oximeter. Specifically, the device 60 can include an ear thermometer 62, which can be any suitable type of ear thermometer, such as an IR ear thermometer. The general construction of the ear thermometer can be typical of traditional ear thermometers, in that the ear thermometer can have a probe portion 64 configured to be disposed in an ear of a subject, and an infrared detector associated with the probe portion to receive radiation from the ear to facilitate determination of a temperature of the subject.

The medical examination device 60 can also include a pulse oximeter 66 integrated with the ear thermometer 62. For example, the pulse oximeter 66 can be associated with the probe portion 64 of the ear thermometer 62. As with other pulse oximeters described herein, the pulse oximeter can have one or more light emitters 68 and one or more photodetectors 70, which can be proximate to one another to measure oxygen saturation of the subject's blood via reflectance. Thus, the pulse oximeter 66 can be integrated with the probe portion 64 and therefore used in a manner typical of the probe portion 64, which is to be inserted into the ear of a subject.

Additionally, the medical examination device 60 can include a display 72 for displaying information of the ear thermometer 62 and/or the pulse oximeter 66. In one aspect, the display 72 can be associated with a handle 74 of the ear thermometer 62. One or more batteries can be included to provide power for the ear thermometer, pulse oximeter, the display, and/or any electronic circuitry, processor, transmitter, receiver, etc. incorporated into the medical examination device. A battery charger can be disposed in, and associated with, a base 76 that can be configured to receive the medical examination device 60 when not in use.

As with other medical examination device examples discussed herein, a timer and an alert feature can be included to facilitate determining a heart rate of the subject by a user. Accordingly, the alert feature can be configured to provide any suitable type of alert, such as an audible alert, a visual alert, and/or a haptic alert.

In one aspect, the medical examination device can be configured to determine a heart rate of the subject by electronic means, such as with one or more electrodes for electrocardiography. The medical examination device can also include any suitable button, switch, touch screen, etc. to facilitate operation and control of the device by a user, such as powering the device on/off, selecting an operation mode, or selecting a display mode.

FIG. 6 illustrates a mount 80 for the medical examination device 60 of FIG. 5 when not in use. It will be noted that a similar mount can also be used to secure or store any other variation of the composite medical examination device described herein. The mount 80 can attach to a mobile stand or caddy used to support a vitals monitor. For example, the mount 80 can attach to the mobile stand directly below the vitals monitor and can be secured to the mobile stand by a clamp 82. Generally, the mount 80 can include a clamp portion 82 having an extension arm 84 which extends away from the clamp portion 82. A device engagement portion 86 can be oriented at a distal end of the extension arm 84. The device engagement portion 86 can be adapted to removably secure the composite medical device 88 to the mount 80. In one aspect, a battery charger can be disposed in, and associated with, the mount.

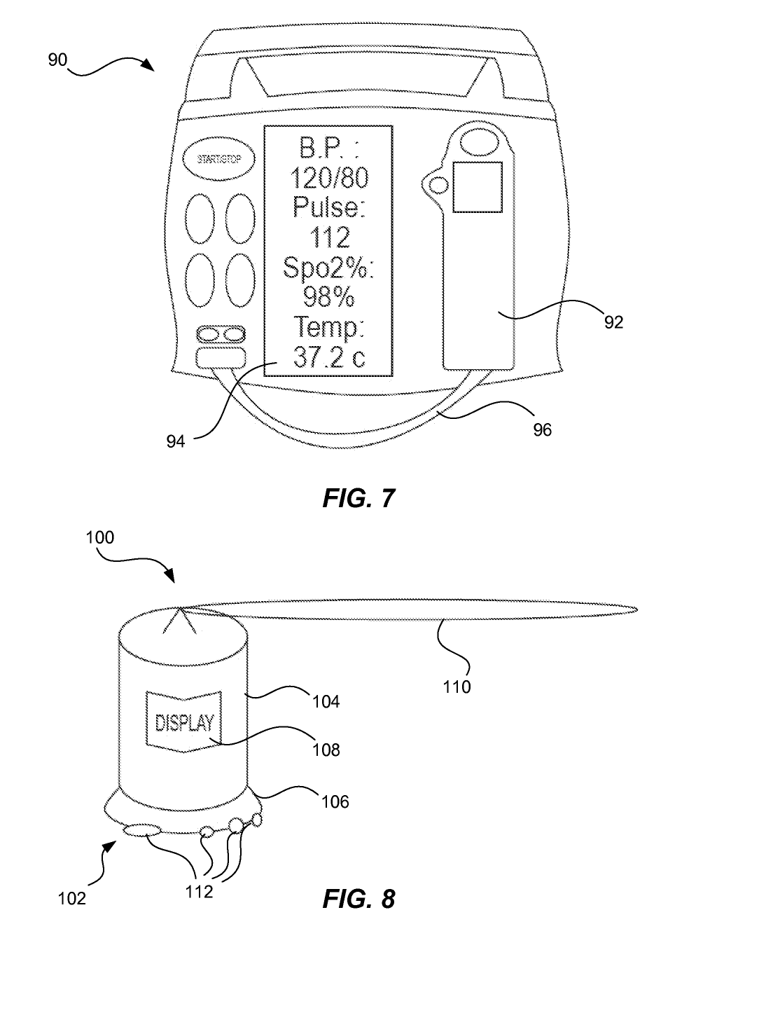

FIG. 7 illustrates a vitals monitor 90 that can be configured to support and/or removably couple with the medical examination device 92 of FIG. 5. For example, when the medical examination device 92 is coupled with the vitals monitor 90, blood oxygen measurements can be displayed on a main screen 94 of the vitals monitor 90. An attachment cable 96 can also facilitate continuous use of the pulse oximeter, such as in a special or intensive care setting. The medical examination device 92 can also be optionally detached from the vitals monitor main unit to provide portability when desired. In one aspect, the vitals monitor 90 can include a built-in cartridge to dispense protective covers for the probe portion of the medical examination device 92.

FIG. 8 illustrates another alternative embodiment which provides a medical examination device 100 having a pulse oximeter 102 integrated into a housing 104. The pulse oximeter 102 also includes a soft diaphragm 106 that conforms to body contours. A display 108 can be integrated into a side wall of the housing 104. Further, an optional attachment loop 110 can be used to allow attachment of the device to the wrist or other locations. The pulse oximeter 102 can include photodiodes, LEDs, and/or photodetectors 112 (as previously described) which are connected to a bottom portion of the housing 104. Thus, the diodes can be fixed to the bottom portion or directly to the diaphragm. The housing and diaphragm are shaped to avoid collection of bacteria such that opposing surfaces, deep contours or other members are avoided. For example, the diaphragm can generally be open-faced and the housing can be cylindrical, spherical, cuboid, or the like.

It is to be understood that the above-referenced embodiments are illustrative of the application for the principles of the present invention. Numerous modifications and alternative arrangements can be devised without departing from the spirit and scope of the present invention while the present invention has been shown in the drawings and described above in connection with the exemplary embodiment(s) of the invention. It will be apparent to those of ordinary skill in the art that numerous modifications can be made without departing from the principles and concepts of the invention as set forth in the claims.

What is claimed is:

1. A medical examination device, comprising:
a stethoscope having a chest piece coupled to at least one ear piece via a yoke, the chest piece having an acoustic portion configured to interface with a subject's body to provide sounds produced in the body to the ear pieces;
a pulse oximeter associated with an oximeter portion of the chest piece, the pulse oximeter having a light emitter and a photodetector to measure oxygen saturation of the subject's blood; and
a display for displaying information of the stethoscope and/or the pulse oximeter.

2. The device of claim 1, wherein the light emitter and the photodetector are proximate one another.

3. The device of claim 1, wherein the oximeter portion is on a side of the chest piece opposite the acoustic portion.

4. The device of claim 1, wherein the display is associated with the yoke.

5. The device of claim 1, wherein the display is associated with the chest piece.

6. The device of claim 1, wherein the display is associated with a flexible member extending between the chest piece and the yoke.

7. The device of claim 1, wherein the photodetector comprises a plurality of photodetectors.

8. The device of claim 1, further comprising a timer and an alert feature to facilitate determining a heart rate of the subject by a user.

9. The device of claim 1, wherein the chest piece further comprises a plurality of electrocardiogram leads to provide heart rhythm information.

10. The device of claim 1, wherein the stethoscope electronically communicates sounds produced in the body to the ear pieces.

11. A medical examination system, comprising:
the medical examination device of claim 1 including a battery to provide power to the medical examination device; and
a storage device having a chamber to receive the medical examination device, and a battery charger to charge the battery of the medical examination device when disposed in the chamber.

12. The system of claim 11, wherein the storage device further comprises an ultraviolet light source operable to sanitize the medical examination device when disposed in the chamber.

13. The system of claim 11, further comprising a support structure for the medical examination device configured to slide into and out of the chamber.

14. The system of claim 11, wherein the medical examination device comprises a plurality of medical examination devices and the chamber comprises a plurality of chambers.

15. A medical examination device, comprising:
an ear thermometer having a probe portion configured to be disposed in an ear of a subject, and an infrared detector associated with the probe portion to receive radiation from the ear to facilitate determination of a temperature of the subject;
a pulse oximeter associated with the probe portion, the pulse oximeter having a light emitter and a photodetector to measure oxygen saturation of the subject's blood; and
a display for displaying information of the ear thermometer and/or the pulse oximeter.

16. The device of claim 15, wherein the light emitter and the photodetector are proximate one another.

17. The device of claim 15, wherein the display is associated with a handle of the ear thermometer.

18. The device of claim 15, further comprising a timer and an alert feature to facilitate determining a heart rate of the subject by a user.

19. The device of claim 15, wherein the photodetector comprises a plurality of photodetectors.

20. A medical examination device, comprising:
a. a housing having a base portion;
b. a pulse oximeter integrated into the base portion and adapted to collect pulse oximetry data;
c. a display integrated into the housing to visually communicate the pulse oximetry data; and
d. a flexible diaphragm associated with the base portion to form a patient contact surface.
